(19) United States
(12) Patent Application Publication
(10) Pub. No.: US 2020/0060957 A1
(43) Pub. Date: Feb. 27, 2020

(54) Dried Rehydratable Composition

(71) Applicant: Lonza Ltd, Visp (CH)

(72) Inventors: Daniel Thorn Leeson, Chatham, NJ (US); Khat Kevin Lou, East Brunswick, NJ (US); Jordan Todorov Petkov, Cheshire (GB)

(73) Assignee: Lonza Ltd

(21) Appl. No.: 16/551,066

(22) Filed: Aug. 26, 2019

Related U.S. Application Data
(60) Provisional application No. 62/723,311, filed on Aug. 27, 2018.

(51) Int. Cl. A61K 8/73; A61K 47/36; A61K 8/04; A61K 9/06; A61K 31/455; A61K 31/375; A61K 8/49; A61K 8/368; A61K 47/12; A61Q 19/00

(52) U.S. Cl. CPC A61K 8/73; A61K 47/36; A61K 8/042; A61K 9/06; A61K 31/455; A61K 31/375; A61K 2800/30; A61K 8/4973; A61K 8/368; A61K 47/12; A61Q 19/00; A61K 2800/26; A61K 8/4926

(57) ABSTRACT

The present disclosure provides a dried rehydratable composition, containing a gelling agent, and a pharmaceutical or cosmetic active ingredient; Further described is a method of rehydrating a dried rehydratable composition, which may be used as a skin benefiting material and may be formulated as a cream, gel, hydrogel, gel or hydrogel patch, mask (including face-mask), lotion, leave-on or rinse-off cosmetic or pharmaceutical product.

### FIELD OF INVENTION

The disclosure relates to a dried rehydratable hydrogel composition effective for cosmetic or pharmaceutical applications.

### BACKGROUND OF THE INVENTION

Agarose is a purified linear galactan hydrocolloid, made up of D-galactose and 3, 6-anhydro-L-galactopyranose, which can be isolated from agar or recovered directly from agar-bearing marine algae. Agarose gels have been used in a variety of applications.

Agarose gels have been previously dried and rehydrated as is described in various patents. Methods of drying and/or rehydrating agarose gels are described in patents, for example, U.S. Pat. No. 5,621,094, U.S. Pat. No. 4,048,377, German published Patent application DE19712708A1, U.S. Pat. No. 4,999,340 and U.S. Pat. No. 3,527,712.

DE19712708A1 describes dried hydrocolloids and/or hydrogels based on polysaccharides, which are prepared using microwave energy and optionally the thermal energy from heated gases or infrared radiation for drying. The products are distinguished by a high product homogeneity, and avoidance of contamination of the dried gel relative to the starting products. Furthermore, dried low-contamination hydrocolloids and/or hydrogels and residual water content is described, which is characterized in that the drying of the hydrocolloids and/or hydrogels under the action of the thermal energy from microwaves.

U.S. Pat. No. 4,999,340 describes a method for separating biological molecules by subjecting the molecules to a separation system in a gel slab. The gel slab is a medium which contains agarose and a linear water-soluble and substantially nonionic polyacrylamide. The dried gel slab is rehydrated using an aqueous separation solvent that will separate biological materials.

U.S. Pat. No. 5,621,094 describes a method of preserving dehydrated agarose gel structure by dehydrating a hydrated agarose gel formed from a mixture comprising agarose and an amount of a non-reducing glycoside of a straight chain sugar alcohol, wherein the agarose gel retains its structure upon rehydration. No active ingredients were suggested to be added to the agarose gel.

U.S. Pat. No. 4,048,377A also describes dried agarose or gelose-containing films which are rehydratable into aqueous gel films containing agarose or gelose and a water-soluble linear polymer or copolymer of acrylamide or methacrylamide. The rehydrated gels are used in immunodiffusion, electrophoresis, immunoelectrophoresis and immunoelectrodiffusion techniques and processes.

U.S. Patent No. 3,527,712A describes a dried rehydratable agarose gel at concentrations of about 0.05% to 5%, which may be dried to provide a dry product that is capable of rehydration to form an aqueous gel. The agarose gel structure formed at concentrations as low as 0.5% to 5% in the presence of the macro-molecular hydrocolloid, which serves as a surrogate, is effective to increase the resistance of the agarose gel to alteration of the pore size or structure thereof during drying and subsequent rehydration of the agarose gel.

Although it has been reported that agarose gels can be dried and rehydrated, the methods described above are highly dependent on the presence of copolymers, additional polysaccharides, sugar alcohols and/or desiccants as essential components in forming a dry rehydratable hydrogel composition with prolonged stability. Additionally, some fail to rehydrate and maintain stability above room temperature. The aim of the present invention is to provide a stable, more efficient composition essentially free of such subsequent components. The present disclosure provides a dried rehydratable hydrogel composition, wherein the composition contains a gelling agent and a cosmetic or pharmaceutical active ingredient and the dried, hydrated hydrogel can deliver the cosmetic or pharmaceutical active agent to the user of the rehydrated hydrogel.

The present disclosure provides a dried rehydratable composition whereby the gelling agent comprises agarose and an active ingredient. Additionally, the composition may be essentially free of macro-molecular hydrocolloids, co-polymers, sugars, and may be very stable even when present as a dried rehydratable composition. The dried composition, which is uniformly opaque in appearance, may be rehydrated with an aqueous solvent, when desired, to provide a rehydrated hydrogel with a clear appearance, where the active ingredient may be uniformly dispersed and dissolved into the hydrogel. Furthermore, in one aspect, the dried rehydratable composition contains a cosmetic or pharmaceutical active ingredient, which is uniformly dispersed and dissolved in the hydrogel when rehydrated with an aqueous solvent. The dried rehydratable composition may be used as a skin benefiting material, for instance, as part of a cosmetic and/or pharmaceutical product. The cosmetic and/or pharmaceutical product may be formulated as a cream, gel, hydrogel, gel or hydrogel patch, mask (including face-mask), lotion, leave-on rinse product, among others.

The present disclosure provides an answer to the need of providing a method of administering an active ingredient in a gelling agent through a dried rehydratable composition and a method of producing the same. Furthermore, in one aspect, the composition exhibits prolonged stability. Additionally, in a further aspect, when rehydrated, the active is uniformly dispersed, forming a clear gel in appearance, upon rehydration from its original opaque dried state.

### SUMMARY OF THE INVENTION

In one aspect, the present disclosure is directed to a dried rehydratable composition containing a gelling agent, and a pharmaceutical or cosmetic active ingredient. The rehydratable composition, when rehydrated, is capable of releasing the pharmaceutical or cosmetic active ingredient to the user of the rehydrated composition.

In one embodiment, the dried rehydratable composition containing the gelling agent, and the pharmaceutical or cosmetic active ingredient are present in the composition in a ratio of the gelling agent to active ingredient in the range of 1,000:1 to 1:100. In a further embodiment, the ratio of the gelling agent to the pharmaceutical or cosmetic active ingredient is in the range of 100:1 to 1:25. In a particular embodiment, the ratio of the gelling agent to the pharmaceutical or cosmetic active ingredient is in the range of 10:1 to 1:15.

In a further embodiment, the dried rehydratable composition contains a gelling agent present in an amount of 0.25% to 99% dry weight, and the active ingredient is present in an amount of 0.005% to 99% dry weight, based on the total weight of the gelling agent and the active ingredient. In a further embodiment, the gelling agent is present in an amount of 5% to 67% dry weight, and the active ingredient is present in an amount of 11% to 83% dry weight, based on the total weight of the gelling agent and the active ingredient dried rehydratable composition.

In one particular embodiment, the gelling agent contains agarose.

In one embodiment, the cosmetic or pharmaceutical active ingredient contains an anti-aging active, anti-wrinkle active, an antioxidant, an anti-acne active, an anti-inflammatory active, an anti-pollutant active, an anti-irritant active, an artificial tanning agent, an antiseptic, an anti-skin atrophy active, an accelerator, an anti-itch agent, an anti-glycation agent, an antiperspirant, a biocidal agent, a cleanser, a cellulite reducing agent, a cell repair agents, a collagen-protecting agent, a collagen stimulating agent, a dark circle reducing agent, a depuffing agent, a deodorant agent, an exfoliating agent, an emollient, an environmental protectant, an epidermal growth factor, a firming agent, a humectant, a hydrating agent, a hair loss suppressing agent, a hair growth inhibitor, a desquamation enzyme enhancer, a keratin material, a lipid, a moisturizing agent, a non-steroidal anti-inflammatory agent, an oil-removing agent, an oil-reducing agent an oil regenerating agent, a probiotic, a prebiotic, a post-biotics, a pore minimizing agent, a photo-protective agent, a protease inhibitor, a skin soothing agent, a skin lightening agent, a sunscreen, a skin barrier enhancing agent, a sebum reducing/stimulating agent, a skin sensate, a skin tightening agent, a skin repair active, a skin whitening agent, a self-tanning agent, a skin texture improving agent, a skin tone improving/evening agent, a skin complexion improving agent, a skin hyperpigmentation reducing agent, a skin rejuvenating agent, a topical anesthetic, a vitamin, a whitening agent or a mixture thereof.

In one particular embodiment, the cosmetic or pharmaceutical active ingredient contains niacin, niacinamide, panthenol, famesol, glucosamine, retinoids, vitamin C (ascorbic acid), vitamin D, caffeine, theobromine, allantoin, alpha-bisabol, phytantriol, magnesium ascorbyl phosphate, ascorbyl glucoside, pyridoxine, pyroglutamic acid, sodium pyroglutamate, amino acids, amino acid derivatives, peptides, proteins, enzymes, salicylic acid, benzoic acid, citric acid, or mixtures thereof.

In another embodiment, the dried rehydratable composition is essentially free of an aqueous solvent.

In a further embodiment, the dried rehydratable composition is essentially free of any copolymers, other polysaccharides, sugar alcohols, desiccant or mixtures thereof.

In yet a further embodiment, the dried rehydratable composition further comprises one or more additional ingredients comprising a viscosity modifier, fragrances, co-surfactants, essential oils, natural plant material and, fats, oils, essential fatty acids, humectants, emollients, biocidal agents, polymers, copolymers, other polysaccharides, sugar alcohols, or desiccant.

In another embodiment, the composition further comprises a biocidal agent present in an amount of 0.25 w/w % to 99 w/w %, based on the dry weight of the total composition. Exemplary biocidal agents include an organic acid, a natural extract, a botanical extract, an essential oil, a salt, an organic compound, or mixtures thereof, and, in one aspect, may be a preservative that is a biocidal agent.

In one embodiment, the dried rehydratable composition is in the form of a finely divided particulate form. In another aspect, the dried rehydratable composition is present in the form of film.

In one particular embodiment, the dried rehydratable composition contains 0.25% to 99% dry weight of a gelling agent, 0.005% to 99% dry weight of an active ingredient, and 0.25 w/w % to 99 w/w % of a preservative based upon the dry weight of the total composition; where the active ingredient is a cosmetic or pharmaceutical active that includes niacinamide, ascorbic acid, or mixtures thereof; and the preservative includes phenoxyethanol, caprylyl glycol, chlorphenesin or mixtures thereof.

In another aspect, the dried rehydratable composition has a uniformly opaque appearance.

In another embodiment, the present disclosure further generally includes a method of forming a rehydrated hydrogel, for use in a cosmetic or pharmaceutical product. The method includes:
a) providing a dried rehydratable composition, such as, for instance, a composition described in the above embodiments and aspects;
b) providing an aqueous solvent; and
c) mixing the aqueous solvent with the dried rehydratable composition to form a rehydrated gel; wherein the rehydrated gel is clear and wherein the active ingredient is uniformly dispersed and dissolved in the rehydrated gel.

Furthermore, in one aspect, the aqueous solvent used in the method of forming a rehydrated hydrogen includes water, aqueous alcohols, ammonia water, acid solutions, salt solutions, water-miscible organic solvents or mixtures thereof.

In one embodiment, the dried rehydratable composition is uniformly opaque in appearance when dry, and upon rehydration, forms a rehydrated gel with a clear appearance, where the active ingredient is present in an amount sufficient to be uniformly dispersed and dissolved in the rehydrated gel to result in a rehydrated gel with a clear appearance.

In yet another embodiment, the present disclosure also describes a skin benefiting composition that includes the dried rehydratable composition.

In a further embodiment, the skin benefiting material may be formulated as a cream, gel, hydrogel, gel or hydrogel patch, face mask, lotion, leave-on, or rinse-off cosmetic or pharmaceutical product.

In another aspect, provided is a wet rehydrated hydrogel containing a composition according to the present disclosure and an aqueous solvent.

These and other aspects will become apparent when reading the detailed description of the invention.

### DETAILED DESCRIPTION

It has now been surprisingly found that a simpler, more stable, dried rehydratable composition may be formed from the combination of a gelling agent and a pharmaceutical or cosmetic active ingredient. Additional components such as copolymers, desiccants, polysaccharides, sugar alcohols are not required to produce a dried rehydratable composition according to the present disclosure, and yet the dried rehydratable composition may be stable with desirable aesthetic properties, mechanical strength, and rheological behavior. This ultimately reduces the manufacturing cost, time and number of ingredients used in the end-user formulations. Additionally, other ingredients such as preservatives may be added, without adversely affecting the properties of the composition. Furthermore, the dried rehydratable composition may have a uniformly opaque appearance, which forms a uniformly clear gel when rehydrated with an aqueous solvent. A dried hydrogel having a cosmetic or pharmaceutical active agent uniformly dispersed and/or dissolved within the gel is also described.

The present disclosure encompasses a dried rehydratable composition containing a gelling agent and a pharmaceutical or cosmetic active ingredient. One unique aspect of the invention is that the composition is essentially free of water and may be essentially free of other copolymers, polysaccharides, sugar alcohols, and/or desiccants. This is surprising, as, previously described, other copolymers, polysaccharides, sugar alcohols, and/or desiccants, are required to keep such a composition dry, stable and rehydratable. Furthermore, the composition according to the present disclosure is very stable over an extended period of time.

Although not necessary, preservatives may be added without disturbing the properties of the dried rehydratable composition. A further advantage is that the dried rehydratable compositions can be prepared, which are opaque in appearance, in the dry form and, when rehydrated, form a clear, uniformly dispersed or dissolved pharmaceutical or cosmetic active ingredient in the rehydrated hydrogel composition. The rehydrated composition may be formulated for end-user body care cosmetic or pharmaceutical products such as skin benefiting agents in the form of creams, gels, hydrogels, patches thereof; masks (including face-mask), lotions, leave-on rinses, among others.

The term “gelling agent” herein, refers to any substance that provides the structural integrity to the gel and holds the water or aqueous solvent inside.

The term “essentially free” herein, refers to a composition containing less than 5% w/w % of a particular substance. Suitably, a composition containing less than 3 w/w % of a particular substance, and in one aspect, less than 1% of a particular substance.

The term “essentially free of a preservative” herein refers to a composition containing less than 0.1% of a preservative.

The term “benefiting material” herein refers to any substance that can improve an aspect of the skin, scalp, hair or nails; thereby providing a cosmetic and/or therapeutic effect to the area of application on the skin, or scalp. Benefiting materials may be a cosmetic active ingredient or a pharmaceutical active agent

The term “cosmetic active ingredient” herein, refers to any substance that may be used to improve the aspect of the skin, hair or nails from a cosmetic standpoint, i.e., appearance, state of hydration and other similar properties.

The term “pharmaceutical active ingredient” herein refers to a substance that may be used to provide a pharmaceutical benefit to the subject using a dehydrated composition of the present invention, once rehydrated, such as treating infections and the like of the skin, hair or nails.

Depending on the skin benefit material contained the dried rehydratable composition, the composition, when rehydrated may have one of the following uses, including but not limited to, hydrating the skin, hair or nails, smoothing fine lines and wrinkles; cosmetically treating acne; firming or softening the skin, strengthening; softening; exfoliating; improving and/or evening skin tone and/or texture; skin, hair or nail lightening; tanning; reducing the appearance of pores; absorbing or controlling secretions; protecting and/or soothing the skin, hair or nails, muscles, aches or pains; reducing puffiness, and/or dark circles; stimulating wound healing; warming, refreshing or cooling the skin, hair or nails; relieving inflammation; brightening the complexion; decongesting; reducing swelling; treating dermatological conditions; cushioning; purifying; fragrant; reducing bacterial or micro-organism growth; healing; repelling insects; removing unwanted hair, dirt, or make-up; and coloring or bleaching the target area, or mixtures thereof, and the like.

In accordance to the present invention, a dried rehydratable composition is described, containing a gelling agent, and a pharmaceutical or cosmetic active ingredient. The weight ratio of the gelling agent to active ingredient is in the range of 1,000:1 to 1:100. The composition formed under any ranges, may be in finely particulate form or may be a solid continuous structure.

Desirably, the range for the weight ratio of the gelling agent and the pharmaceutical or cosmetic active ingredient can be further narrowed to encompass other ratios such as about 100:1 to 1:25, such as 10:1 to 1:15; such as 1:1 to 1:10, or such as 1:2 to 1:5.

The dried rehydratable composition as described may have a uniformly opaque appearance. The dried rehydratable composition maybe in a fine particulate form or may a single continuous structure.

Typically, the dried rehydratable composition comprises a gelling agent present in an amount of 0.25% to 99% dry weight, and the active ingredient is present in an amount of 0.005% to 99% dry weight, based on the total weight of the gelling agent and the pharmaceutical or cosmetic active ingredient.

Suitably, the composition comprises a gelling agent present in an amount of 1.5% to 94% dry weight, and the active ingredient is present in an amount of 0.9% to 95% dry weight, based on the total weight of the gelling agent and the active ingredient.

Desirably, the dried rehydratable composition comprises a gelling agent present in an amount of 5% to 67% dry weight, and the active ingredient is present in an amount of 11% to 83% dry weight, based on the total weight of the composition, including the gelling agent and the active ingredient and any other ingredient which may be present.

Such a composition may be later dried, stored and later used in cosmetic or pharmaceutical applications.

Examples of gelling agents include, but are not limited to, polyvinyl alcohol, sodium polyacrylate, acrylate polymers and copolymers with an abundance of hydrophilic groups, hydrogels, hydrophilic polymers capable of forming a highly cross-linked network, or mixtures thereof and the like. Natural gelling agents may also be used, including but not limited to agarose, methylcellulose, hyaluronan, elastin like polypeptides and other naturally derived polymers, or mixtures thereof and the like. Example of an exemplary gelling agent includes agarose.

The dried rehydratable composition, further contains a cosmetic or pharmaceutical active ingredient including, but not limited to, an anti-aging, anti-wrinkle, antioxidants, anti-acne, anti-irritant, anti-inflammatory, anti-pollutant, artificial tanning agents, antiseptics, anti-skin atrophy actives, accelerators, anti-itch agents, anti-glycation agents, antiperspirant, biocidal agent (such as anti-microbial, antibacterial, antiviral, anti-fungal actives, disinfectants, mixtures thereof, and the like), cleansers, cellulite reducing agent, cell repair agents, collagen-protecting agents, dark circle reducing agent, depuffing agent, deodorant agent, exfoliating agents, emollients, environmental protectant, firming agent, humectants, hydrating agents, hair loss suppressing agent, hair growth inhibitors, epidermal growth factors, desquamation enzyme enhancers, keratin materials, lipids, moisturizing agents, non-steroidal anti-inflammatory agents, oil-removing, oil-reducing or oil regenerating agents, probiotics, prebiotics or postbiotics, pore minimizing agent, photoprotective agents (such as organic or mineral UV screening agent), protease inhibitors, skin soothing, skin lightening, sunscreens, skin barrier enhancing agent, sebum reducing/stimulating agent, skin sensates, skin tightening agents, skin repair actives, skin whitening, self-tanning agents, skin texture improving, skin tone improving/evening, skin complexion improving, skin hyperpigmentation reducing, skin rejuvenating, topical anesthetics, vitamins, whitening agents, or mixtures thereof and the like.

Exemplary pharmaceutical or cosmetic active ingredient include, but are not limited to, plant extracts such as botanical extracts, algae extract, mulberry root extract, sweet almond seed extract, Polygonum cuspidatum root extract, Quercus suber extract, and the like; acid compounds; base compounds; caprylyl glycol; 2-ethylhexyl p-methoxycinnamate; 2-ethylhexyl N,N-Dimethyl-p-aminobenzoate; avobenzone; aminopropyl ascorbyl phosphate; benzoyl peroxide; carbomer; caffeine; dihydroxyacetaone; glycerin; hinokitol; hydroquinone and derivatives (e.g. arbutin); magnesium ascorbyl phosphate; mannose phosphate; N-acetyl glucosamine; niacin and derivatives thereof; oxybenzone; octylmethoxycinnamate; phospholipids; palmitoyl oligopeptide; palmitoyl tetrapeptide-7; proteinases and peptidases; probiotics/postbiotics (such as Bifidobacterium, Brevibacterium, Propionibacterium, Lactococcus, Streptococcus, Staphylococcus (e.g., S. epidermidis), Lactobacillus (e.g., L. acidophilus), Enterococcus, Pediococcus, Leuconostoc, or Oenococcus, and mixtures thereof, ferments thereof or lysates thereof); pullulan, teprenone; trisodium resveratrol triphosphate; trehalose; tyrosine; tyrosine estersoctyl methoxylcinnamat; butyl methoxybenzoylmethane; oxybenzone; titanium dioxide; urea, vitamins (such as vitamin A (retinol), vitamin E (tocopherol), vitamin B5 (panthenol), vitamin B3 (niacinamide), and their derivatives (such as esters, biotin or biotin esters)); whey protein, zinc oxide; essential oils; plant cell, microbial and mammalian cell extracts; cell lysates and cultured media; or mixtures thereof and the like.

Examples of pharmaceutical actives, include but are not limited to, retinoids such as retinol, retinoic acid, retinyl proprionate, retinyl palm itate, retinaldehyde, adapalene) and derivatives thereof (e.g. cis and trans); vitamin E, tocopherols and derivatives thereof (such as alpha-tocopherol, beta-tocopherol, gamma-tocopherol, delta-tocopherol, tocopherol acetate, tocopherol nicotinate); resorcinols and derivatives thereof; amino acids, amino acid derivatives, acids or bases, proteins, peptides) and the like; sulfur-containing D and L amino acids and derivatives and salts thereof, for example N-acetyl derivatives thereof; palmitoyl pentapeptide or argireline; enzymes (such as palmityl penta-peptide-3, pitera, mevastatin, lovastatin, palmitoyl-lysine-threonine, palmitoyl-lysine-threonine-threonine-lysine-serine, N-undecyl-10-enoyl-L-phenylalanine); butylated hydroxytoluene (BHT); triclosan, erythromycin, tolnaftate, nystatin or clortrimazole; topical analgesics, such as benzocaine, lidocaine or procaine; hydrocortisone, clobetasol, dexamethasone, or prednisone; mixtures thereof and the like.

Examples of acids compounds include, but are not limited to, organic and/or inorganic acids or acidic functional groups, e.g., carboxyl groups, phenolic hydroxyl groups; such as and, 1-hydroxy-2-naphthoic acid, 2,2-dichloroacetic acid, 2-hydroxyethanesulfonic acid, 2-oxoglutaric acid, 4-acetamidobenzoic acid, 4-aminosalicylic acid, acetic acid, acetyl salicylic acid, adipic acid, ascorbic acid (L), aspartic acid (L), benzenesulfonic acid, benzoic acid, camphoric acid (+), camphor-10-sulfonic acid (+), capric acid (decanoic acid), caproic acid (hexanoic acid), caprylic acid (octanoic acid), carbonic acid, cinnamic acid, citric acid, cyclamic acid, dodecylsulfuric acid, ethane-1,2-disulfonic acid, ethanesulfonic acid, ferulic acid, formic acid, fumaric acid, galactaric acid, gentisic acid, glucoheptonic acid (D), gluconic acid (D), glucuronic acid (D), glutamic acid, glutaric acid, glycerophosphoric acid, glycyrrhizic acid or glycyrrhetic acid, hippuric acid, hyaluronic acid, hydrobromic acid, hydrochloric acid, isobutyric acid, isolinoleic acid, kojic acid, lactobionic acid, lauric acid, linoleic acid, maleic acid, malic acid (−L), malonic acid, mandelic acid (DL), methanesulfonic acid, naphthalene-1,5-disulfonic acid, naphthalene-2-sulfonic acid, nicotinic acid, nitric acid, oleic acid, oxalic acid, p-aminobenzoic acid, palmitic acid, pamoic acid, phosphoric acid, propionic acid, pyroglutamic acid (−L), salicylic acid or derivatives thereof (such as 5-octanoylsalicylic acid and 4 methoxysalicylic acid), sebacic acid, stearic acid, succinic acid, sulfuric acid, tartaric acid (+L), thiocyanic acid, toluenesulfonic acid (p) and undecylenic acid, salts thereof, or mixtures thereof, and the like.

Example of bases include, but are not limited to, hydroxides or oxides of alkali metals or alkaline earth metals, a-amino carboxylic acids having an additional amino group, carbonates or bicarbonates of alkali metals or alkaline earth metals, acetates or formates of alkali metals and alkaline earth metals, or mixtures thereof and the like. Suitable bases include sodium hydroxide, potassium hydroxide, sodium carbonate and potassium carbonate.

Examples of essential oils include, but are not limited to, balsam oil, pine oil, citrus oil, lime oil, jasmine oil, rose oil, muscatel sage oil, chamomile oil, lavender oil, clove oil, melissa oil, mint oil, cinnamon leaf oil, lime blossom oil, Wa cholderbeeröl, vetiver oil, olibanum oil, galbanum oil and labdanum oil orange blossom oil, neroli oil, orange peel oil, sandalwood oil, anise oil, aniseed oil, arnica blossom oil, basil oil, bay oil, Chamberlain pacablütenöl, silver fir oil, noble fir cone oil, elemi oil, eucalyptus oil, fennel oil, pine needle oil, galbanum oil, geranium oil, ginger grass oil, Guaj akholzöl, Gurj unbalsamöl, helichrysum oil, Ho oil, ginger oil, iris oil, cajeput oil, calamus oil, camomile oil, camphor oil, Kanagaöl, cardamom oil, cassia oil, pine needle oil, Kopalvabalsamöl, coriander oil, spearmint oil, caraway oil, Kuminöl, lemongrass oil, ambrette seed oil, myrrh oil, mustard seed oil, clove oil, neroli oil, niaouli oil, olibanum oil, oregno oil, palmarosa oil, patchouli oil, petitgrain oil, pepper oil, peppermint oil, pimento oil, pine oil, rose oil, rosemary oil, sandalwood oil, celery oil, sternanis oil, thuja oil, thyme oil, verbena, vetiver oil, wormwood oil, wintergreen oil, ylang-ylang oil, hyssop oil, cinnamon oil, cinnamon leaf oil, and cypress oil, or mixtures thereof and the like.

Particular examples of a cosmetic or pharmaceutical ingredient that may be used includes, but are not limited to, niacin, niacinamide, panthenol, famesol, glucosamine, retinoids, vitamin C (ascorbic acid), vitamin D, caffeine, theobromine, allantoin, alpha-bisabol, phytantriol, magnesium ascorbyl phosphate, ascorbyl glucoside, pyridoxine, amino acids, pyroglutamic acid or salts thereof (i.e. sodium pyroglutamate), amino acid derivatives peptides, proteins, enzymes, glycolic acid, lactic acid, salicylic acid, benzoic acid, citric acid, or mixtures thereof, and the like.

In a particular embodiment, the present invention provides a dried rehydratable composition which is essential free of an aqueous solvent. “Aqueous solvent” is defined as the aqueous solvent is water, aqueous alcohols, ammonia water, acid solutions, salt solutions, water-miscible organic solvents in water or mixtures thereof.

In a further embodiment, the dried rehydratable composition is essentially free of any copolymers, other polysaccharides, sugar alcohols, desiccant or mixtures thereof and the like.

The present dried rehydratable composition further comprises a biocidal agent present in an amount of 0.25 w/w % to 99 w/w %, based on the dry weight of the total composition. The biocidal agent may also act to preserve the rehydratable dried composition during storage.

The amount of a biocidal agent used may vary depending on the active ingredient (cosmetic or pharmaceutical) used in the dried rehydratable composition. It may be desirable to also contain a dried rehydratable composition which is essentially free of a preservative. Typically the biocidal agent may be present in an amount between 1.5% to 94% dry weight; such as 5 w/w % to 67 w/w % dry weight, of the dried rehydratable composition.

Suitably, the biocidal agent is a preservative comprising an organic acid, botanical extract, essential oil, salt, organic compound, or mixtures thereof, and the like. Some of the beneficial agents may also function as a preservative, thereby removing the need of a preservative in addition to the beneficial agents.

Examples of preservatives include, but are not limited to, natural acids and their salts thereof (such as citric, cinnamic, tartaric, malic, oxalic, ascorbic acids); organic acids, sorbic acid, citric acid, benzoic acid, salicylic acid and their salts, (i.e. potassium sorbate, sodium benzoate); isothiazolones (such as CMIT/MIT, MIT, BIT, OIT); hydantoins (i.e. DMDMH); ethylhexylglycerin; capryl glycol, phenyl ethanol, phenyl propanol, phenoxyethanol, benzyl alcohol; bronopol, triclosan; dehydroacetic acid, imidazolidinyl urea, PHMB, chlorhexidine, diazolidinyl urea, chlorphenesin, sodium hydroxymethylglycinate, benzethonoium chloride, IPBC (iodopropynyl butylcarbamate), essential oil, or combinations thereof, and the like.

Exemplary preservatives include ascorbic acid, isothiazolones, essential oils, salicylic acid, phenoxyethanol, caprylyl glycol & chlorphenesin, salts thereof, or mixtures thereof, and the like.

In one aspect, the provided is a dried rehydratable composition comprising 0.25% to 99% dry weight of a gelling agent, 0.005% to 99% dry weight of an active ingredient, and 0.25 w/w % to 99 w/w % of a preservative; where the active ingredient is a cosmetic or pharmaceutical active. In a particular embodiment, the gelling agent is agarose. In another particular embodiment, the cosmetic or pharmaceutical active agent is niacinamide, ascorbic acid, or mixtures thereof. In addition, the preservative, in a particular embodiment, is phenoxyethanol, caprylyl glycol, chlorphenesin or mixtures thereof; based on the weight of the total composition.

In another aspect, the present disclosure provides a dried rehydratable composition that includes a gelling agent, present in an amount of 1.5% to 94% dry weight, and 0.9% to 95.5% dry weight of an active ingredient, based on the total weight of the gelling agent and the active ingredient; and 1.5 w/w % to 94 w/w % of a preservative that includes phenoxyethanol, caprylyl glycol, chlorphenesin or mixtures thereof; based on the weight of the total composition; where the active is a cosmetic or pharmaceutical active that includes niacinamide, ascorbic acid, or mixtures thereof.

In another aspect, the dried rehydratable composition may include 5 w/w % to 67 w/w % dry weight of agarose gel; 11 w/w % to 83 w/w % dry weight of niacinamide and 5 w/w % to 67 w/w % dry weight of a preservative.

A further aspect of the current disclosure describes a method of forming a rehydrated composition, for use in a cosmetic or pharmaceutical product. In one aspect, the method includes:
a) providing a dried rehydratable composition according to the present disclosure;
b) providing an aqueous solvent; and
c) mixing the two components together to form a gel; where the rehydrated gelling agent and the active ingredient are uniformly dispersed and dissolved in the clear gel.

Accordingly, the present disclosure also describes a method of forming a rehydrated composition, for use in a cosmetic or pharmaceutical product by:
a) providing a dried rehydratable composition according to the present disclosure;
b) providing an aqueous solvent;
c) mixing the two components together to form a gel; where the gel is clear and where the active ingredient is dissolved in the clear gel;
   where the composition further contains a biocidal agent; where the biocidal agent is a preservative present in an amount between 0.25 w/w % to 99 w/w %, based on the dry weight of the total composition.

In one aspect, compositions can be prepared where the active ingredient is present in an amount sufficient to yield a dried rehydratable composition that is uniformly opaque in appearance and upon rehydration yields a gel which is clear in appearance containing uniformly dispersed and/or dissolved active.

Accordingly, upon rehydration, the dried rehydrated composition forms a clear gel, where the active ingredient is uniformly dispersed and dissolved in the clear gel.

Additionally, the ratios of the gelling agent and the active ingredient are maintained within the ranges as previously described. For example, upon rehydration of a dried rehydratable composition, the ratio of the gelling agent and the active ingredient in the wet gel may be in a range of about 100:1 to 1:25, such as 10:1 to 1:15, such as 1:1 to 1:10, or such as 1:2 to 1:5.

Furthermore, another aspect of the present disclosure describes a method for forming a rehydrated composition having a uniform, clear gel appearance by:
a) providing the dried rehydratable composition as described in the present disclosure;
b) providing an aqueous solvent; and
c) mixing the two components together to form a gel; where the resulting rehydrated gelling agent is uniformly clear and where the active ingredient is uniformly dispersed and dissolved in the clear gel.

Examples of aqueous solvents include, but are not limited to, water, aqueous alcohols, ammonia water, acid solutions, salt solutions, water-miscible organic solvents, polyhydroxy alcohols (such as glycerin, diglycerin, propylene glycol, dipropylene glycol, sorbitol, pantenol and sugar); urea, alpha-hydroxy acid and its salt; and low molecular weight polyethylene glycols with molecular weight less than 20,000; water soluble or dispersible polymers; or mixtures thereof and the like. Suitably the aqueous solvent comprises water.

Furthermore, in another aspect the present disclosure provides a wet rehydrated hydrogel that includes a composition according to the present disclosure and an aqueous solvent.

The ingredients used in the compositions of the present disclosure may further include one or more additional ingredients including, but not limited to, a viscosity modifier, fragrances, co-surfactants, essential oils, natural plant material and, fats, oils, essential fatty acids, humectants, emollients, biocidal agents, polymers, copolymers, other polysaccharides, sugar alcohols, or desiccant, and the like.

Additionally, the present disclosure also generally describes a skin benefiting composition that includes the dried rehydratable composition according to the present disclosure.

The skin benefiting composition includes a skin benefiting material; whereby the skin benefiting material may be formulated as a cream, gel, hydrogel, gel or hydrogel patch, mask (including face-mask), lotion, leave-on rinse cosmetic or pharmaceutical products, or mixtures thereof, and the like.

The following examples illustrate the invention without limitation. All parts and percentages are given by weight unless otherwise indicated.

It will be understood that each of the elements described in the examples below, or two or more together may also find a useful application in other types of methods differing from the type described above. Without further analysis, the foregoing will so fully reveal the gist of the present disclosure that others can, by applying current knowledge, readily adapt it for various applications without omitting features that, from the standpoint of prior art, fairly constitute essential characteristics of the generic or specific aspects of this disclosure set forth in the appended claims.

The foregoing embodiments are presented by way of example only; the scope of the present disclosure is to be limited only by the following claims.

### Example 1

TABLE 1
Dried rehydratable composition

| Ingredient | % | % | % | % |
|---|---|---|---|---|
| Water | q.s to 100 | q.s to 100 | q.s to 100 | q.s to 100 |
| Agarose | 1.00 | 1.00 | 1.00 | 1.00 |
| Niacinamide | 5.00 | — | — | — |
| Arabinogalactan | — | 3.00 | — | — |
| Vitamin C | — | — | 2.00 | — |
| Salicylic Acid | — | — | — | 2.00 |
| Preservative | 0.75 | 0.75 | 0.75 | 0.75 |
| Water | 93.25 | 95.25 | 96.25 | 96.25 |
| Total | 100.00 | 100.00 | 100.00 | 100.00 |

The table above presents the composition of active ingredients and hydrogel used to prepare a dried rehydratable composition. Each composition was prepared by dispersing agarose in room temperature water using constant agitation, and then the sample was heated to 85° C. with agitation, until all particles are dissolved. The samples were cooled to 55° C., then poured into a petri dish (hot) at desired thickness and allowed to reach room temperature and some alternatively stored at 45° C. until dried.

### Example 2

TABLE 2
Results of producing dried rehydratable compositions

| Ingredient | Wet Gel | Dried Gel | Observation |
|---|---|---|---|
| Niacinamide | ✓ | ✓ | Opaque |
| Niacinamide* | ✓ | ✓ | Opaque |
| pyroglutamic acid | ✓ | ✓ | Clear, slightly sticky |
| Pyroglutamic | ✓ | ✓ | Opaque |
| Acid, Niacinamide | | | |
| arabinogalactan | ✓ | ✓ | Clear |
| Vitamin C | ✓ | ✓ | Slight discoloration, apparent |
| | | | crystallization of Vitamin C |
| | | | upon drying |
| Salicylic Acid | ✓ | ✓ | Opaque |

*See Example 3.

Results from each composition showed that only agarose exhibited a clear gel upon rehydration of the uniformly opaque dried state. In this clear gel form, the active ingredient is uniformly dispersed and dissolved within the gel, in comparison to the other actives that were prepared, which formed opaque gels upon rehydration, as shown in Table 2. Niacinamide and salicylic acid formed a uniformly opaque gel.

### Example 3

TABLE 3
Formulations with niacinamide.

| Ingredient | # 1% | # 2% |
|---|---|---|
| Water | q.s to 100 | q.s to 100 |
| Glycerin | 2.00 | 2.00 |
| L-Pyroglutamic Acid | 2.50 | 2.50 |
| Niacinamide | — | 3.50 |
| Agarose | 0.75 | 0.75 |
| Ethanol | 30.00 | 30.00 |
| Sodium Hydroxide 50% | 2.00 | 2.00 |
| Total | 100.00 | 100.00 |

The formulation was prepared by combining the ingredients as shown in Table 3. The inclusion of ethanol accelerates drying; and the inclusion of glycerin promotes more homogenous drying and results in a dry gel that is more structurally flexible (i.e. it can be rolled similar to that of paper, whereas the dry gel without glycerin is more prone to break).

While the invention has been described above with references to specific embodiments thereof, it is apparent that many changes, modifications and variations can be made without departing from the invention concept disclosed herein. Accordingly, it is intended to embrace all such changes, modifications, and variations that fall within the spirit and broad scope of the appended claims.

What is claimed is:

1. A dried rehydratable composition comprising a gelling agent, and a pharmaceutical or cosmetic active ingredient; and wherein the weight ratio of the gelling agent to active ingredient is in the range of 1,000:1 to 1:100.

2. The dried rehydratable composition according to claim 1, wherein the gelling agent is present in an amount between 0.25%-99% weight, and the pharmaceutical or cosmetic active ingredient is present in an amount between 0.005%-99% weight, based on the total weight of the gelling agent and the active ingredient.

3. The dried rehydratable composition according to claim 2, wherein the gelling agent is present in an amount between 5%-67% weight, and the pharmaceutical or cosmetic active ingredient is present in an amount between 11%-83% dry weight, based on the total weight dried rehydratable composition.

4. The rehydratable composition comprising according to claim 1, wherein the gelling agent comprises agarose.

5. The dried rehydratable composition according to claim 1, wherein the cosmetic or pharmaceutical active ingredient comprises an anti-aging active, anti-wrinkle active, an antioxidant, an anti-acne active, an anti-inflammatory active, an anti-pollutant active, an anti-irritant active, an artificial tanning agent, an antiseptic, an anti-skin atrophy active, an accelerator, an anti-itch agent, an anti-glycation agent, an antiperspirant, a biocidal agent, a cleanser, a cellulite reducing agent, a cell repair agents, a collagen-protecting agent, a collagen stimulating agent, a dark circle reducing agent, a depuffing agent, a deodorant agent, an exfoliating agent, an emollient, an environmental protectant, an epidermal growth factor, a firming agent, a humectant, a hydrating agent, a hair loss suppressing agent, a hair growth inhibitor, a desquamation enzyme enhancer, a keratin material, a lipid, a moisturizing agent, a non-steroidal anti-inflammatory agent, an oil-removing agent, an oil-reducing agent an oil regenerating agent, a probiotic, a prebiotic, a post-biotics, a pore minimizing agent, a photo-protective agent, a protease inhibitor, a skin soothing agent, a skin lightening agent, a sunscreen, a skin barrier enhancing agent, a sebum reducing/stimulating agent, a skin sensate, a skin tightening agent, a skin repair active, a skin whitening agent, a self-tanning agent, a skin texture improving agent, a skin tone improving/evening agent, a skin complexion improving agent, a skin hyperpigmentation reducing agent, a skin rejuvenating agent, a topical anesthetic, a vitamin, or a mixture thereof.

6. The dried rehydratable composition according to claim 5, wherein the cosmetic or pharmaceutical active ingredient comprises niacin, niacinamide, panthenol, famesol, glucosamine, retinoids, vitamin C (ascorbic acid), vitamin D, caffeine, theobromine, allantoin, alpha-bisabol, phytantriol, magnesium ascorbyl phosphate, ascorbyl glucoside, pyridoxine, amino acids, pyroglutamic acid, sodium pyroglutamate, amino acid derivatives peptides, proteins, enzymes, glycolic acid, lactic acid, salicylic acid, benzoic acid, citric acid, or a mixture thereof.

7. The dried rehydratable composition, according to claim 1, wherein the composition is essential free of an aqueous solvent.

8. The dried rehydratable composition according to claim 1, wherein the composition is essentially free of any copolymers, other polysaccharides, sugar alcohols, or desiccants.

9. The dried rehydratable composition according to claim 1, wherein the ratio of the gelling agent to the pharmaceutical or cosmetic active ingredient is 100:1 to 1:25.

10. The dried rehydratable composition according to claim 9, wherein the ratio of the gelling agent to the pharmaceutical or cosmetic active ingredient is 10:1 to 1:15.

11. The dried rehydratable composition, according to claim 1, further comprises one or more additional ingredients comprising a viscosity modifier, a fragrance, a co-surfactant, an essential oil, a fat, an oils, an essential fatty acid, a humectant, an emollients, a biocidal agent, a sugar alcohol, or a desiccant.

12. The dried rehydratable composition, according to claim 1, further comprises a biocidal agent present in an amount between 0.25 w/w % - 99 w/w %, based on the dry weight of the total composition.

13. The dried rehydratable composition, according to claim 12, wherein the biocidal agent is a preservative comprising an organic acid, natural extract, botanical extract, essential oil, salt, organic compound, or mixtures thereof.

14. The dried rehydratable composition, according to claim 13, wherein the preservative comprises an ascorbic acid, isothiazolones, essential oils, salicylic acid, phenoxyethanol, caprylyl glycol & chlorphenesin, natural extracts, salts thereof, or mixtures thereof.

15. The dried rehydratable composition, according to claim 1, wherein the composition is in finely divided particulate form.

16. The dried rehydratable composition, according to claim 1, wherein the composition is uniformly opaque in appearance.

17. A dried rehydratable composition comprising 0.25%-99% weight of a gelling agent, 0.005%-99% weight of an active ingredient, and 0.25 w/w %-99 w/w % of a preservative; wherein said active ingredient is a cosmetic or pharmaceutical active comprising niacinamide, ascorbic acid, or a mixture thereof; and said preservative comprises phenoxyethanol, caprylyl glycol, chlorphenesin or a mixture thereof; based on the weight of the total composition.

18. The dried rehydrated composition according to claim 1, wherein the dried rehydratable composition has uniformly opaque in appearance when dried and, upon rehydration with an aqueous solvent, the active ingredient uniformly dispersed and dissolved in the rehydrated gel that the rehydrated gel has a clear appearance.

19. A method of forming a rehydrated composition, for use as a cosmetic or pharmaceutical product, said method comprises:
a) providing a dried rehydratable composition according to claim 1;
b) providing an aqueous solvent; and
c) mixing the aqueous solvent with the dried rehydratable composition together to form a rehydrated gel; wherein the rehydrated gel is clear and wherein the active ingredient is uniformly dispersed and dissolved in the rehydrated gel.

20. The method of forming a rehydrated composition according to claim 19, wherein the aqueous solvent comprises water, aqueous alcohols, ammonia water, acid solutions, salt solutions, water-miscible organic solvents or mixtures thereof.

21. A wet rehydrated hydrogel comprising dried rehydratable composition according to claim 1, and an aqueous solvent.

22. A skin benefiting composition comprising the dried rehydratable composition according to claim 1.

23. The skin benefiting composition according to claim 22, wherein the skin benefiting material is formulated as a cream, gel, hydrogel, gel or hydrogel patch, face mask, lotion, leave-on or rinse-off cosmetic or pharmaceutical product.
